(19) United States
(12) Patent Application Publication
(10) Pub. No.: US 2007/0059382 A1
(43) Pub. Date: Mar. 15, 2007

(54) Medical treatment of breast cancer with boric acid materials

(75) Inventors: Stephen Carper, Las Vegas, NV (US); Susan Meacham, Henderson, NV (US)

Correspondence Address: Mark A. Litman & Associates, P.A., 3209 West 76th St., York Business Center, Suite 205, Edina, MN, 55435, US

(73) Assignee: Board of Regents, Univ. and Comm. College System of Nevada...

(21) Appl. No.: 11/519,116

(22) Filed: Sep. 11, 2006

Related U.S. Application Data
(60) Provisional application No. 60/715,984, filed on Sep. 9, 2005.

(51) Int. Cl. A61K 33/22

(52) U.S. Cl. 424/658

(57) ABSTRACT

Breast cancer cell lines selected from the group consisting of ZR-75-1 and SK-BR-3 are treated by a method comprising: identifying the presence of at least one of breast cancer cell lines ZR-75-1 and SK-BR-3 in a patient; and providing a solution of boric acid or boric acid salts to the patient in a manner that delivers boric acid or boric acid salt to breast cancer cells in the patient.

### RELATED APPLICATIONS DATA

This application claims priority from U.S. Provisional Application 60/715,984 filed Sep. 9, 2005.

### BACKGROUND OF THE INVENTION

1. Field of the Invention

The present invention relates to the field of cancer prevention and treatment and particularly the treatment and prevention of breast cancer in human beings.

2. Background of the Art

Carcinoma is the term used to describe most malignant tumors. Most breast cancers are ductal carcinomas—they originate in the ducts that carry milk to the nipple. Less common are lobular carcinomas. These form in the cells that line the lobules that produce milk. Tumors that originate in bone, muscle, fat, or connective tissue are called sarcomas. Sarcomas of the breast are very rare. Much less common types of tumors also include tubular, medullary, mucinous, papillary, and adenocystic tumors.

If the cancer cells are confined to the duct or lobule, the cancer is in situ, meaning it hasn't left the site. Ductal carcinoma in situ (DCIS) is usually found by mammography, as no tumor mass has formed and, as a result, a woman couldn't find the cancer during breast self-examination. When a cancer has moved beyond the duct, it is called invasive or infiltrating cancer. Infiltrating ductal carcinoma is the most common type of breast cancer. As the cells invade surrounding areas, scar tissue or other fibrous growth surrounds the tumor cells forming a lump that can be seen on a mammogram or felt during a physician's examination.

Infiltrating lobular carcinoma doesn't produce the same kind of fibrous growth, so it may be harder to detect on a mammogram. This type of cancer produces a softer lump—sometimes it is describes as a thickening. If a lobular cancer is found in one breast, it may also be in the other breast. Therefore it is important to carefully monitor the second breast.

Currently, medical castration using hormone drugs is widely used for cancer therapy, but there are cancers which do not respond to hormone drugs (hormone-independent cancer). Also in hormone-dependent cancers exhibiting effects in hormone therapy, it is known that when therapy is continued, hormone-dependent cancers change into hormone-independent cancers by proliferation of hormone-independent cancers.

Therefore, in therapy for cancers (in particular, prostate cancer, breast cancer, etc.), it is ideal that drugs are properly used according to a type of cancer such as cancer which responds to hormone therapy and cancer which does not respond to hormone therapy, a stage of cancer disease, an age, etc. of an individual patient.

Apoptosis refers to, for example, cell shrinkage, chromatin condensation, nucleus concentration, disappearance of microvillus on the cell surface, plasma membrane blebbing, apoptotic body formation, gap between peripheral cells accompanied with cell shrinkage, and removal by phagocytes. Apoptosis or programmed cell death plays an important role in individual development and homeostasis maintenance in a living body. It has been gradually made clear that abnormality of apoptosis causes diseases such as cancers, autoimmune diseases and nervous diseases.

There are many cancer patients who do not respond to therapy by hormone drugs, and in such cancer patients, proliferation of hormone-independent cancer cells is caused by use of hormone drugs, and effects of treatment of cancers by hormone drugs are not continued.

It is desired to develop anticancer agents which are excellent in effects of treating or preventing cancers or their metastasized lesions, or recurred cancers and have no side effects.

Boronate enzyme inhibitors have wide application, from detergents to bacterial sporulation inhibitors to pharmaceuticals. In the pharmaceutical field, there is patent literature describing boronate inhibitors of serine proteases, for example thrombin, factor Xa, kallikrein, elastase, plasmin as well as other serine proteases like prolyl endopeptidase and Ig AI Protease. Thrombin is the last protease in the coagulation pathway and acts to hydrolyse to four small peptides form each molecule of fibrinogen, thus deprotecting its polymerisation sites. Once formed, the linear fibrin polymers may be cross-linked by factor XIIIa, which is itself activated by thrombin. In addition, thrombin is a potent activator of platelets, upon which it acts at specific receptors. Thrombin also potentiates its own production by the activation of factors V and VIII. Other aminoboronate or peptidoboronate inhibitors or substrates of serine proteases are described in: [0018] U.S. Pat. No. 4,935,493; WO 94/25049; WO 95/09859; U.S. Pat. Nos. 4,450,105; 5,106,948; 5,169,841. Peptide boronic acid inhibitors of hepatic C virus protease are described in WO 01/02424.

Adams et al, U.S. Pat. No. 5,780,454 (1998); U.S. Pat. No. 6,066,730 (2000); U.S. Pat. No. 6,083,903 (2000); U.S. Pat. No. 6,297,217 (2001) describe peptide boronic ester and acid compounds useful as proteasome inhibitors. These documents also describe the use of boronic ester and acid compounds to reduce the rate of muscle protein degradation, to reduce the activity of NF-kappaB in a cell, to reduce the rate of degradation of p53 protein in a cell, to inhibit cyclin degradation in a cell, to inhibit the growth of a cancer cell, to inhibit antigen presentation in a cell, to inhibit NF-kappaB dependent cell adhesion, and to inhibit HIV replication. Brand et al, WO 98/35691, teaches that proteasome inhibitors, including boronic acid compounds, are useful for treating infarcts such as occur during stroke or myocardial infarction. Elliott et al, WO 99/15183, teaches that proteasome inhibitors are useful for treating inflammatory and autoimmune diseases.

U.S. Pat. No. 6,696,419 (Miljkovic) describes that inflammation is affected by topical application of a boron containing compound/complex in which a central tetrahedral boron atom is covalently bound to four ligands. At least one of the ligands preferably includes an oxygen, nitrogen, carbon, or sulfur atom, and preferably all four ligands include at least one such atom. Preferred ligands are saccharides and amino acids, including fructose, sorbitol, mannitol, xylitol, sorbose, serine and threonine. Especially preferred ligands have a conformation with at least two hydroxyl groups, or one hydroxyl group and one amino group in a 1,2- and a 1,3-position relative to each other, providing a high association constant in the range of about 3,000 and about 20,000. The compounds/complexes are preferably provided in formulations which provide good transdermal delivery, including appropriate solvent systems, microemulsions, liposomes. Particularly targeted inflammations are those of the joints and skin, including bums such as sunburn.

U.S. Pat. No. 5,312,816 (Spielvogel) describes a method of combating, e.g., preventing as well as treating, a disease state such as cystic fibrosis, neonatal hypoxemia, pulmonary hypertension, adult respiratory distress syndrome, psoriasis, spondyloarthritis, rheumatoid arthritis, gout, inflammatory bowel disease, myocardial infarctions, and/or osteoporosis in an animal subject, by administering to the animal subject an effective amount of an organic boron compound. The organic boron compounds usefully employed in the method of the invention include any suitable organic boron-containing compounds, such as Lewis base-boron adducts; a preferred class of organic boron compounds useful in such method includes boron analogs of .alpha.-amino acids, and the corresponding amides and esters of such amino acids. A method is also disclosed of inhibiting enzyme activity in in vitro or in vivo systems comprising administering to such system an enzyme-inhibiting amount of an organic boron compound. Further disclosed is a method of reducing hydroxyproline, calcium, and/or inorganic phosphorous in serum and/or urine of an animal subject, by administering to the animal subject an effective amount of an organic boron compound.

It is known that derivatisation of boronic adds as cyclic esters provides oxidation resistance. For example, U.S. Pat. No. 5,681,978 (Matteson D S et al) teaches that 1,2-diols and 1,3 diols, for example pinacol, form stable cyclic boronic esters that are not easily oxidised.

Various disclosures of medication and treatment compositions may include adjuvants such as buffers in addition to the primary medically active ingredients. For example, Published U.S. Patent Application 2005/0163807 describes that pharmaceutical compositions, including the specific compositions of the Application, may further contain various additives such as buffers, isotonizing agents and chelating agents. Examples of usable buffers include boric acid, phosphoric acid, acetic acid, citric acid, epsilon.-aminocaproic acid, glutamic acid, and/or their corresponding salts (e.g., alkali metal or alkaline earth metal salts, such as sodium salts, potassium salts, calcium salts and magnesium salts). Examples of usable isotonizing agents include sodium chloride, potassium chloride, saccharides and glycerol. Examples of usable chelating agents include sodium edetate and citric acid.

20040235748 (Igari etal.) teaches that the GnRH agonist or antagonist include GnRH agonists or antagonists effective against hormone dependent diseases, in particular, sex hormone dependent cancers (e.g. prostate cancer, uterus cancer, breast cancer, pituitary tumor etc.), and sex hormone dependent diseases such as prostatic hypertrophy, endometriosis, uterine myoma, precocious puberty, dysmenorrhea, amenorrhea, premenstrual syndrome and polycystic ovary syndrome, and for contraception (or sterility in the case where a rebound effect after stopping administration is utilized). Additional examples of the GnRH agonist or antagonist include GnRH agonists or antagonists effective against benign or malignant tumors or the like which are sex hormone independent, but GnRH sensitive. The reference also teaches that boric acid salts of these agonists and antagonists may be effective.

The term “cancer” or “cancer cell” is used herein to denote a tissue or cell found in a neoplasm which possesses characteristics which differentiate it from normal tissue or tissue cells. Such characteristics include but are not limited to degree of anaplasia, irregularity in shape, indistinctness of cell outline, nuclear size, changes in structure of nucleus or cytoplasm, other phenotypic changes, presence of cellular proteins indicative of a cancerous or pre-cancerous state, increased number of mitoses, and ability to metastasize. Words pertaining to “cancer” include carcinoma, sarcoma, tumor, epithelioma, adenoma, leukemia, lymphoma, polyp, scirrus, transformation, neoplasm, and the like.

The term “neoplastic”, when referring to cells, indicates cells undergoing new and abnormal proliferation, particularly in a tissue wherein the proliferation is uncontrolled and progressive, resulting in a neoplasm. The neoplastic cells can be either malignant, i.e. invasive and metastatic, or benign.

Individuals are encouraged to eat a greater proportion of fruits and vegetables to decrease their risk of developing cancer. The mechanism whereby this increased dietary intake inhibits cancer development is an active area of investigation. While most research has focused on the organic compounds found in plants, a universal preventative agent has not been identified. Recently, it has been reported that boron, an essential mineral in plants, inhibits growth in specific cell lines of prostate cancer. Preliminary results in our laboratory indicate that boron inhibits growth in specific breast cancer cell lines. Boron is a required nutrient in all higher plants. Since plants are the single largest source of boron in the human diet, boron represents a common factor in fruits and vegetables that may prevent/inhibit cancers in humans. The major goal of this project is to investigate the molecular mechanism whereby boron kills breast cancer cells.

The major form of boron in the body is boric acid B(OH)₃, which is a weak acid with a pK=9.2. Therefore, at physiological pH most of the boric acid (BA) is unionized. Boron is found in drinking water with concentrations ranging from low (0.1 mg B/L) to very high (29 mg B/L) depending on the proximity of the water source to borax deposits. The primary sources of boron in the human diet are fruits and vegetables. The average American diet provides approximately 1-2 mg of boron per day. Boric acid is typically excreted in the urine within 24 hrs of ingestion.

Boric acid inhibits the growth of prostate cancer in selected cell lines: DU-145, PC-3 and LNCaP but not non-tumorigenic prostate cell lines. Boric acid also caused a dose-dependent reduction in cyclins A-E and MAPK proteins, and reduced adhesion, migration and invasion potential in DU-145 cells. No mechanism was proposed for how boric acid could have such a wide range of effects. Boric acid can inhibit the growth of LNCaP cells in nude mice. The mechanism for this growth inhibition may have been dependent upon the ability of boron to inhibit prostate-specific antigen (PSA), a serine protease that can cleave insulin-like growth factor binding protein-3 to produce IGF-1 leading to tumor growth. However, PSA inhibition does not explain the boric acid effects on the DU-145 cells since these cells do not express PSA. A limited epidemiological study indicated that boron may inhibit prostate cancer in men. Taken together, these studies provide strong evidence that boric acid can prevent/inhibit prostate cancer growth in humans.

The terms “treat” and “treatment” are used broadly to denote therapeutic and prophylactic interventions that favorably alter a pathological state. Treatments include procedures that moderate or reverse the progression of, reduce the severity of, prevent, or cure a disease. In the case of cancers, treatment includes an increase in survival rate over a given time period or an increase in survival time, reduction in tumor mass, reduction in tumor metastasis, cessation of disease progression, reduction in time to progression, and the like.

All references cited above and herein are incorporated by reference to assist in enablement of the present invention and for background information

### SUMMARY OF THE INVENTION

It has been determined that the provision of solutions of boric acid to at least some breast cancer cell lines can inhibit the growth of those breast cancer cell lines. This practice has been proven in growth media and can be extrapolated to human in vivo introduction for treatment.

### BRIEF DESCRIPTION OF THE FIGURES

FIG. 1 shows a graph of data relating to treatment of the MCF7 breast cancer cell line with a 1 mM solution of boric acid.

FIG. 2 shows a graph of data relating to treatment of the T47D breast cancer cell line with a 1 mM solution of boric acid.

FIG. 3 shows a graph of data relating to treatment of the ZR-75-1 breast cancer cell line with a 1 mM solution of boric acid.

FIG. 4 shows a graph of data relating to the treatment of the SK-BR-3 breast cancer cell line with a 1 mM solution of boric acid.

FIG. 5 shows a graph of data relating to the treatment of the MD MBA 231 breast cancer cell line with a 1 mM solution of boric acid.

FIG. 6 shows a graph of data relating to the treatment of the MD MBA 435 breast cancer cell line with a 1 mM solution of boric acid.

FIG. 7 shows data comparing ZR-75-1 with cells floating and cells attached.

FIG. 8 shows graphs of efficacy with two lines over days time periods.

### DETAILED DESCRIPTION OF THE INVENTION

Solutions of boric acid (e.g., solutions of at least 0.025, at least 0.05 and at least 1 mM, which last is equal to 1000 μM boric acid), when added to the growth media of cells can inhibit the growth rate of several types of breast cancer cell lines. Although the literature contains several reports that boric acid (which is the major form of boron in the blood) can inhibit the growth of human prostate cancer cell lines, and specifically that DU-145, PC3 and LNCaP show different sensitivities to boric acid solutions added to their media (Barranco, WT and Eckhert CD. Boric acid inhibits human prostate cancer cell proliferation. Cancer Lett. 216:21-29, 2004), this is not indicative of the effectiveness of boric acid towards other cancer cell lines. These same prostate cell lines can form tumors in mice. The growth of the LNCaP tumor is also inhibited (about 30%) in nude mice if they are treated with low levels of boric acid (1.7 and 9.0 mgB/Kg/day) (Gallardo-Williams, M T, Chapin, R E, King, P E, Moser, G J, Goldsworthy, T L, Morrison, J P and Maronpot, R R. Boron supplementation inhibits the growth and local expression of IGF-1 in human prostate adenocarcinoma (LNCaP) tumors in nude mice.

No one knows the exact mechanism whereby boron inhibits tumor cell growth. It is known that boron can bind to cis diols which are present in many sugar molecules, steroid hormones and some nucleotides, but there has been no connection between this binding phenomena and growth inhibition. It is also known that boron acid can reversibly inhibit serine proteases. How this enzyme inhibition would account for growth inhibition is also unknown at this time (Hunt, C D and Idso J P. Dietary boron as a physiological regulator of the normal inflammatory response: a review and current research progress. Trace Elem. Exper. Med. 12:221-233, 1999).

While boric acid does not ionize at physiological pH it is capable of forming a stable covalent complex through esterification with cis-diol containing compounds, especially ribose containing molecules including: NAD⁺, NADH, NADP⁺, NADPH, SAM, and nucleotide mono, di- and triphosphates. Boric acid is also capable of binding to six carbon sugars including: glucose, fructose, mannitol and galactose. Boron can enter the mammalian cell either by direct transport via a boron transport protein NaBC1 or by passive diffusion across the cell membrane. Boron binding to membrane lipids can result in several changes including an increase in membrane fluidity and a decrease in membrane hydration. Not surprisingly, boron has a greater affinity for lipids containing polyhydroxylated moieties (e.g. galactolipids and phosphatidyllinositol). These differential interactions may result in various boron-induced modulations of membrane-associated processes in the cell.

Cells form attachments to other cells and to the extracellular matrix using four major classes of adhesion molecules: Cadherins, which are calcium dependent cell to cell binding molecules that bind to other cadherins; Selectins, which in the presence of calcium bind weakly to specific oligosaccharides on another cell; Immunoglobulin superfamily members including ICAM (intercellular adhesion molecules); and Integrins, which can bind to other cell molecules or to the extracellular matrix. Integrins are a family of non-covalent heterodimeric cell surface receptors. There are 18 α and 8 β subunits that combine to form at least 24 αβ heterodimer. Each subunit has a large (>700 residue) NH₂-terminal extracellular domain. A single membrane-spanning domain links this extracellular domain to a generally short (13-70 residue) cytoplasmic domain.

Most cells express more than one type of integrin heterodimer. Integrin expression profiles are unique for distinct cell types, and can change with developmental stage and physiological conditions. With the exception of the fibronectin receptor α5β1, all integrins bind to more than one ligand. Each extra-cellular matrix (ECM) molecule is also bound by more than one integrin. At sites of integrin activation and clustering, protein aggregates termed focal complexes and focal adhesions assemble on the intracellular surface. The types of proteins that form these complexes can be grouped as either structural, which form links to the actin cytoskeleton, or signaling. The signaling complex includes a variety of kinases and adapter molecules linking integrins to other kinases, GTPase family members, phospholipases and ion channels.

Integrin expression in human breast cancer cell lines has been well studied. The boric acid sensitive breast cancer cell line ZR-75-1 expresses: alpha2beta1, alpha3beta1, alpha6beta4 and alphaVbeta1 integrins, as do the MCF-7, MDA-MB-23 1, MDA-MB-435 and T47D cell lines which are not growth inhibited by boric acid. The MCF-7 cell line, in addition to the four integrins listed above, also expresses: alpha4beta1, alpha5 beta1, alphaVbeta3, alphaVbeta5, alpha6beta1 and alpha7beta1. The expression of these six additional integrins may provide some additional adhesion sites and explain the lack of sensitivity to boric acid. Since the literature has not yet reported a unique integrin expressed in ZR-75-1 cells, the level of expression of the different integrins may play a crucial role in boric acid sensitivity. A recent report demonstrated that the prostate cancer cell line DU-145 (which are sensitive to BA) express several beta1 integrins including alpha1beta1 but no similar reports for prostate cell lines LNCaP and PC3 (which are much less sensitive to BA) have occurred.

Integrin signaling can involve several different pathways depending on the specific integrin involved and the adaptor molecules expressed by the cell. Integrin signaling results in phosophorylation of the focal adhesion kinase (FAK) and the integrin-linked kinase (ILK) which can activate several pathways including those that stimulate apoptosis: phosphatidyl inositol 3-kinase/PKB (Akt) cascade and JNK or those that inhibit apoptosis: Ras/ERK/MAP kinase cascade (21) and activation of PKC and p53.

Apoptosis can be induced by various agents and treatments including the loss of cell anchorage to a substrate. This form of apoptosis termed anoikis, has been reported in numerous cell types including mammary epithelial cells and prostate epithelial cells. Integrins play a central role in anoikis, for example if the αᵥβ3 integrin, which binds vitronectin, is blocked cells will undergo anoikis. Several studies have indicated that activation of caspase 8 is a common feature of anoikis. When boric acid is placed on chicken cartilage or human fibroblasts it causes cells to release/secrete proteins including tumor necrosis factor α (TNFα). TNFα0 can induce apoptosis or stimulate proliferation depending on the type of receptors and adaptor molecules expressed by the cancer cell.

Integrins are transmembrane proteins and hence glycosylated. Based on the amino acid sequences, integrin α5β3 contains 14 potential asparagines-linked glycosylation sites on the α chain and 12 such sites on the β chain. Modulation of these sugar moieties can reduce the affinity of the integrin for binding to ECM proteins. In K562 human erythroleukemia cells, treatment with N-glycosidase F, (an amidase that cleaves between the inner most GlcNAc and asparagines residues of N-glycans from N-linked glycoproteins), resulted in blocking α5β1 binding to fibronectin and prevented the association of both integrin subunits with each other. These results demonstrated that the sugar residues on the integrins play key roles in both association of the α and β subunits as well as the ability of the integrins to bind to ECM proteins.

Integrins can exist in two conformational states: an active, ligand binding state and an inactive state. The equilibrium between these two states can be regulated by external and internal molecules including metal ions such as Mg²⁺, Mn²⁺, and Ca²⁺. X-ray studies of integrins have revealed the ligand “head” of the integrin contains a seven-blade β-propeller fold in the α subunit and a von Willebrand factor type A-domain in the β subunit (βA-domain). Cation-binding sites are present on the lower face of the β-propeller domain and the upper face of the βA-domain. One site on the β chain known as the MIDAS (metal ion-dependent adhesion site) plays a critical role in ligand binding. Once a divalent metal ion is bound (coordinated through the carboxylate oxygen of an Asp residue) at the MIDAS site a conformational change occurs and the integrin is now capable of binding a ligand (43). The amino acid residues that play a role in the MIDAS site in β₃ are Asp¹¹⁹, Ser¹²¹, Ser¹²³ and Asp²¹⁷ (44). A similar MIDAS sequence is present in β⁷ chain indicating that this sequence is conserved in all the beta chains. The two serine residues in the MIDAS site could provide OH moieties necessary for binding to boron. Hence boron could compete with divalent metal cations for the MIDAS site.

Certain integrins are continually internalized from the plasma membrane into endosomal compartments and then recycled back to the cell surface. The rate of integrin internalization/recycling from the plasma membrane pool through the endosomal system occurs at least once every 30 minutes. The αᵥβ5 integrin is recycled through clathrin-coated pits but integrins of the β1 and β3 classes are internalized by mechanisms that depend on dynamin, PKC-α and caveolin-1. Once internalized, the integrins are delivered to early endosomes where they will either be processed for degradation or returned to the plasma membrane. The return to the plasma membrane can occur through two separate mechanisms: a short loop and a long loop. In the short loop, integrins are sorted to particular subdomains of the early endosomes and returned to the plasma membrane under control of Rab4 GTPase with a t_{1/2} of 3 minutes. In the long loop, integrins are passed to the perinuclear recycling compartment and then returned to the plasma membrane by a Rab11 dependent mechanism with a t_{1/2} of 10 minutes. Disruption of integrin recycling can lead to a decrease in the rate of migration or even detachment of cells.

Boric acid has the potential to interact at many sites both on the surface as well as inside the cell. Preliminary studies presented in the next section indicate that boric acid and phenyl boric acid 1 are capable of causing breast cancer cells to detach and undergo apoptosis 2 inhibit the attachment of ZR-75-1 cells. Both of these responses are controlled by integrins.

The present technology has developed because of the independent studies by the inventors investigating the ability of boric acid to inhibit the growth of breast cancer cell lines. The first four breast cancer cell lines we investigated showed no growth inhibition when treated with a 1 mM solution of boric acid. The cell lines were MDA-MB-23 1, MDA-MB-435, (both of which are estrogen receptor negative) and MCF-7, T-47D (both of which are estrogen receptor positive). The studies were extended to two more human breast cancer cell lines: ZR-75-1 (estrogen receptor positive) and SK-BR-3 (estrogen receptor negative), both of which display a growth inhibition in the presence of 1 mM boric acid. Upon treatment of the cells for three days with boric acid and then removal of the boric acid, the cell lines resumed their normal growth rate after about three days following the boric acid removal. Other cell lines that offer potential for similar treatment according to the practices of the technology described herein are included in the list included within the APPENDIX attached hereto, which is a part of this application.

The present technology includes a method for the treatment of breast cancer cell lines, any cell line identified as responding to the particular treatment described, but especially selected from the group consisting of ZR-75-1 and SK-BR-3, the method comprising: identifying the presence of at least one of breast cancer cell lines ZR-75-1 and SK-BR-3 (or others responsive to the described treatment) in a patient; and providing a solution of boric acid or boric acid salts to the patient in a manner that delivers boric acid or boric acid salt to breast cancer cells in the patient. The solution may comprise at least 0.05 or at least 0.10 mM of boric acid or boric acid salt. The method may be practiced, for example, wherein the solution is delivered intravenously or injected to a site where breast cancer cells have been located within the patient.

The following is some background on Boron. Boron is the fifth element in the period chart of the elements. The primary form of boron in the body is boric acid B(OH)₃. Hunt (1998) has shown that boron can act as an inhibitor for a wide variety of enzymes in vitro, but no boron-containing enzyme has been reported (Hunt, C D. Regulation of enzymatic activity. One possible role of dietary boron in higher animals and humans. Biol Trace Elem Res. 66:205-225, 1998).

Boron enters the human body through the water supply as well as foodstuffs and supplements. Foods that are high in boron include: avocado, peanut butter, peanuts, prune and grape juice, chocolate powder, wine, pecans, and granola raisin and raisin bran cereals (Meacham S L, and Hunt C D. Dietary boron intakes of selected populations in the United States. Biol Trace Elem Res. 66:65-78, 1998). Coffee and milk, while normally low in boron, may be major contributors in humans due to the large volumes of these liquids that are typically consumed (Culver B D, Hubbard S A. Inorganic boron health effects in humans: An aid to risk assessment and clinical judgment. J Trace Elem Exp Med 9:175-184,1996). Human and animal studies report that about 90% of the consumed boron is absorbed. The boron is converted to boric acid in the gut. Boric acid typically has a half-life of about 21 hrs before most (about 85%) of it is excreted in the urine. Since there is no know function for boron in humans there is no Recommended Dietary Allowance or Adequate intake level established by the government. In NHANES III (National Health and Nutrition Examination Study), the median consumption of boron ranged from 0.75 to 0.96 mg/day for school-aged children and from 0.87 to 1.35 mg/ day for adults. The adult median boron intake from supplements was approximately 0.14 mg/day. Therefore, the median intake of dietary and supplemental boron was approximately 1.0 to 1.5 mg/ day for adults.

There is a Tolerable Upper Intake Level (UL) established for boron in the book entitled “Dietary Reference Intakes for Vitamin A, Vitamin K, Arsenic, Boron, Chromium, Copper, Iodine, Iron, Manganese, Molybdenum, Nickel, Silicon, Vanadium, and Zinc (2000)” which is a product of the Food and Nutrition Board of the Institute of Medicine (IOM) of the National Academy of Sciences. The level calculated from developmental data in the rat and extrapolated to a 61 Kg average female human was 20 mg/day. The Tolerable Upper Intake Level (UL) is the highest level of daily nutrient intake that is likely to pose no risk of adverse health effects for almost all individuals. A report by Stokinger (1981) listed the minimal lethal dose of boric acid from ingestion is 640 mg/kg/day (Stokinger HE. 1981. The halogens and nonmetals boron and silicon. In: Clayton G D, Clayton F E, eds. Patty's Industrial Hygiene and Toxicology, Vol. 2B. New York: John Wiley and Sons. Pp. 2978-3005). Symptoms of boron toxicity include nausea, gastric discomfort, vomiting, and diarrhea. At higher doses, skin flushing, excitation, convulsions, depression, and vascular collapse have been reported. Borates have been used to treat epilepsy at doses between 1,000 mg/day of boric acid (2.5 mg/kg/ day) to 25 g/day of boric tartrate (24.8 mg/kg/day) were administered chronically, toxicity was expressed as dermatitis, alopecia, anorexia, and indigestion (Culver B D, Hubbard S A. Inorganic boron health effects in humans: An aid to risk assessment and clinical judgment. J Trace Elem Exp Med 9:175-184,1996). No adverse effects in humans at chronic intakes of 2.5 mg/kg/day (about 1 g of boric acid) were reported by Culver and Hubbard (supra).

Human blood levels have been reported to range between 13 to 70 μM (Ward, NL. The determination of boron in biological materials by neutron irradiation and prompt gamma-ray spectrometry. J. Radioanal. Nucl. Chem. 110:633-639, 1987). This variation is most likely due to dietary intakes. In Turkey, drinking water boron concentrations of 2683 μM have not been shown to cause any deleterious effects in humans exposed over many generations (Sayli, B S, Tuccar, E., and Elhan A H. An assessment of fertility in boron-exposed Turkish subpopulations. Rep. Toxicol. 12:297-304, 1986). If 000 μM boron is needed in the blood to inhibit breast cancer growth (it might take less in an animal model or in humans, we would need to do these studies) and the typical human has 5 L of blood, then it would be desirable to add 310 mg of boron/day to achieve this level. This would appear to be a dose larger then the UL suggested by the IOM, but far less than that consumed in Turkey. The IOM recommendation of 20 mg/day would result in a boron blood concentration of 65 μM, which is within the normal range.

In the practice of the present technology, although boric acid and boric acid salts are preferred compounds, alternative borates, boranes and boron compounds could include organic boron compounds which may find utility in the broad practice of the present invention are the following specific boron-containing organic compounds: (1) Trimethylamine-borane; (2) t-Butylamine-borane; (3) Dimethylamine-borane; (4) Morpholine-borane; (5) Diethylamine-borane; (6) Pyridine-borane; (7) Triphenylphosphine-borane; (8) Ammonia-borane; (9) Ammonia-cyanoborane; (10) Methylamine-cyanoborane; (11) Dimethylamine-cyanoborane; (12) Trimethylamine-cyanoborane; (13) Triphenylphosphine-cyanoborane; (14) Triethylphosphite-cyanoborane; (15) 2′-Deoxycytidine-N3-cyanoborane; (16) 2′-Deoxyadenosine-N1-cyanoborane; (17) N-methylmorpholine-cyanoborane; (18) Ethylenediamine-bis(cyanoborane); (19) N,N,N′,N′-Tetramethylethylenediamine-bis(cyanoborane); (20) Morpholine-carboxyborane; (21) Triphenylphosphine-carboxyborane; (22) Trimethylamine-carboxyborane; (23) Ammonia-carboxyborane; (24) Triethylphosphite-carboxyborane; (25) N,N-Dimethyloctadecylamine-carboxyborane; (26) N,N-Dimethylhexadecylamine-carboxyborane; (27) Trimethylamine-carboethoxyborane; (28) Trimethylamine-carbomethoxyborane; (29) Trimethylamine-carbobenzoxyborane; (30) Methylamine-carbomethoxyborane; (31) Dimethylamine-carbomethoxyborane; (32) Ammonia-carbomethoxyborane; (33) N,N-Dimethylhexadecylamine-carbomethoxyborane; (34) N,N-Dimethyloctadecylamine-carbomethoxyborane; (35) Ammonia-N-ethylcarbamoylborane; (36) Methylamine-N-ethylcarbamoylborane; (37) Dimethylamine-N-ethylcarbamoylborane; (38) Trimethylamine-N-ethylcarbamoylborane; (39) Trimethylamine-N-propylcarbamoylborane; (40) Trimethylamine-N-phenylcarbamoylborane; (41) Trimethylamine-N-octylcarbamoylborane; (42) 2-(Acetoxy)ethyldimethylamine-borane; (43) 2-(Thioacetoxy)ethyldimethylamine-borane; (44) 2-(Hydroxy)ethyldimethylamine-borane; (45) Diethyl((N,N-Dimethylamine)methyl)phosphonate-N-cyanoborane; (46) Trimethylamine-methyldicyanoborane; (47) Trimethylamine-isopropyldicyanoborane; (48) Trimethylamine-boranecarbohydroxamic acid tetraphenylborate salt; (49) [(Trimethylamine-boryl)carbonyl]glycine methyl ester; (50) [(Trimethylamine-boryl)carbonyl]phenylalanine methyl ester; (51) [(Trimethylamine-boryl)carbonyl]tyrosine methyl ester; (52) [(Trimethylamine-boryl)carbonyl]serine methyl ester; (53) [(Trimethylamine-boryl)carbonyl]methionine methyl ester; (54) [(Ammonia-boryl)carbonyl]valine methyl ester; (55) [(Ammonia-boryl)carbonyl]isoleucine methyl ester; (56) [(Ammonia-boryl)carbonyl]leucine amide; (57) Pyridine-carboxyborane; (58) N-Methylpyridine-carboxyborane; (59) N-Cyanopyridine-carboxyborane; (60) Trimethylamine-cyanomethylborane; (61) Trimethylamine-2-cyanoisopropylborane; (62) Trimethylamine-.alpha.-cyanobenzylborane; (63) Trimethylamine-carboxyborane, sodium salt; and (64) Dimethylamine-carboxyborane, sodium salt. In general, carboxyborane adducts of Lewis bases (e.g., boron analogs of .alpha.-amino acids) and amide and ester derivatives thereof are a preferred class of organic boron compounds in the practice of the present invention, and metal complexes of such Lewis base-carboxyborane adducts and their amide and ester derivatives may also be usefully employed.

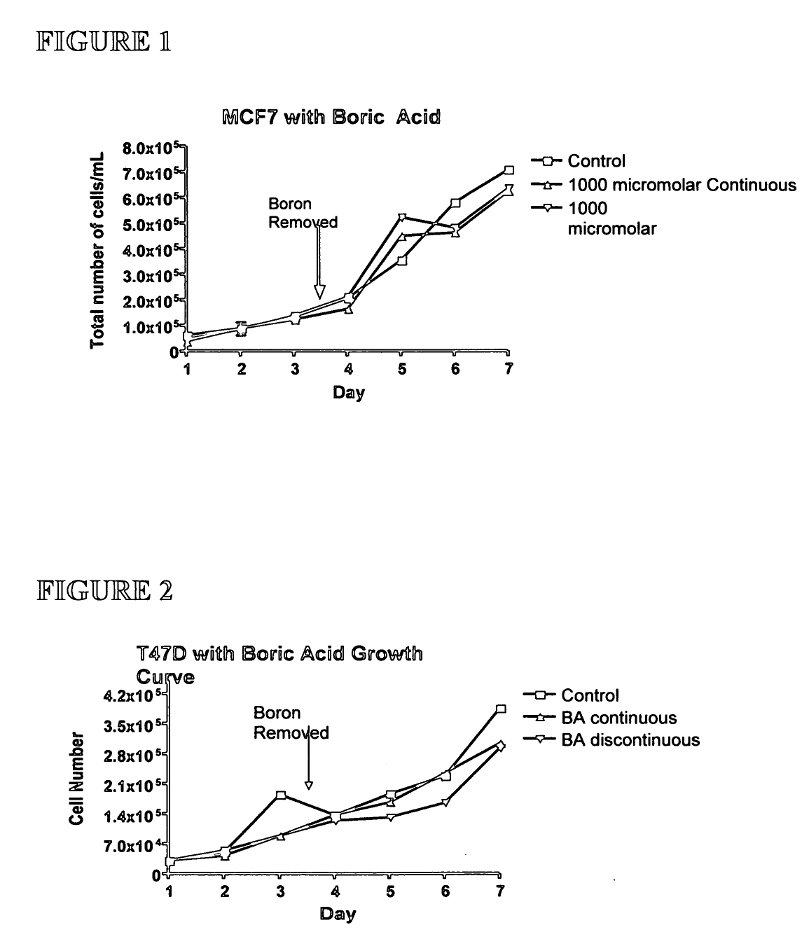

FIG. 1 shows a graph of data relating to treatment of the MCF7 breast cancer cell line with a 1 mM solution of boric acid.

FIG. 2 shows a graph of data relating to treatment of the T47D breast cancer cell line with a 1 mM solution of boric acid.

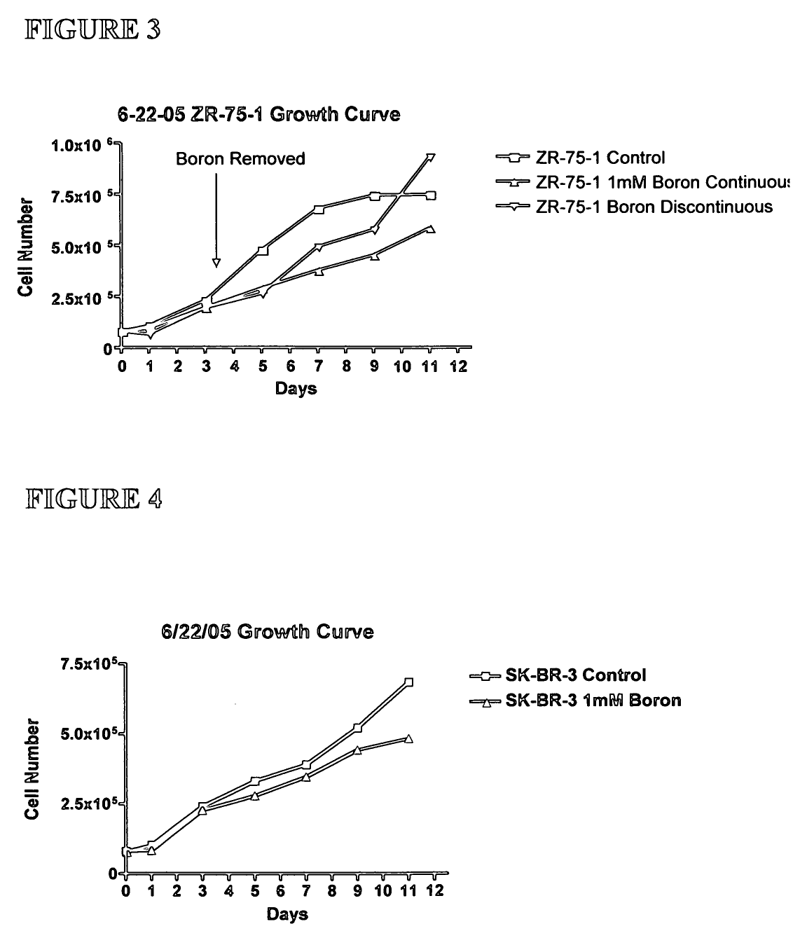

FIG. 3 shows a graph of data relating to treatment of the ZR-75-1 breast cancer cell line with a 1 mM solution of boric acid.

FIG. 4 shows a graph of data relating to the treatment of the SK-BR-3 breast cancer cell line with a 1 mM solution of boric acid.

FIG. 5 shows a graph of data relating to the treatment of the MD MBA 231 breast cancer cell line with a 1 mM solution of boric acid.

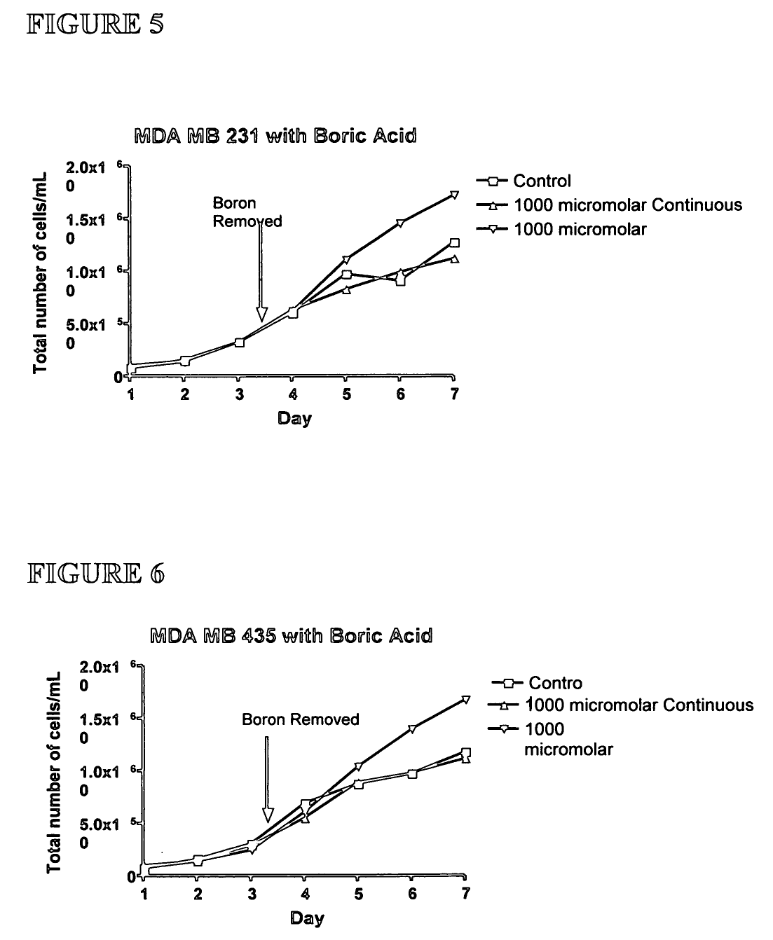

FIG. 6 shows a graph of data relating to the treatment of the MD MBA 435 breast cancer cell line with a 1 mM solution of boric acid.

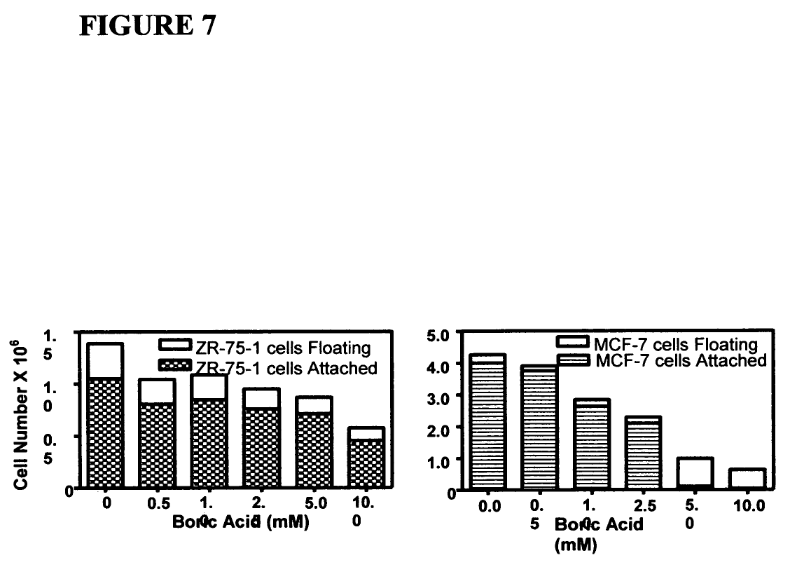

The ability of the treated cells to begin to grow after the removal of boric acid would indicate that boric acid is acting as a cytostatic agent. However, when cells were treated for seven days in the presence of boric acid in concentrations ranging from 0.5 to 10 mM cells were seen floating in the media, a potential sign of anoikis. We quantified the attached and floating cells. The data is shown below in FIG. 7.

Adjuvants may be present with the boron or borates when they are introduced into the patient, or may be added relatively prior to or relatively subsequent to the main or initial addition of borates or boron to the patient. For example, the protein, NaBC1, is found in most tissues and is part of a large family of ion transporting proteins that allow charged molecules to travel across cell membranes. Ion transporters are embedded in the cell membrane, opening and closing like gates to let charged ions and molecules enter and leave the cell. The movement of these molecules affects numerous essential cellular functions. Like other nutrients, cells must transport boron across the membrane to control its concentration within the cell. The discovery of NaBC1 may help cells control internal boron concentration and the role of boron in a wide range of cellular processes, such as cell growth, bone mineralization and cancer treatment according to the presently disclosed technology. “NaBC1 is very specific for the transport of borate. BOR1 has also been found to be a boron transporter and may be used in this capacity also.

Thus, the present invention may be practiced with the boron compounds being provided in pharmaceutical formulations, both for veterinary and for human medical use, comprising the active agent (the organic boron compound) together with one or more pharmaceutically acceptable carriers thereof and optionally any other therapeutic ingredients. The carrier(s) must be pharmacutically acceptable in the sense of being compatible with the other ingredients of the formulation and not unsuitably deleterious to the recipient thereof. The active agent is provided in an amount effective to achieve the desired pharmacological effect, as described above, and in a quantity appropriate to achieve the desired daily dose.

The formulations include those suitable for oral, rectal, topical, nasal, ophthalmic, or parenteral (including subcutaneous, intramuscular, and intravenous) administration. Formulations suitable for parenteral administration are preferred.

The formulations may conveniently be presented in unit dosage form and may be prepared by any of the methods well known in the art of pharmacy. All methods include the step of bringing the active compound into association with a carrier which constitutes one or more accessory ingredients. In general, the formulations may be prepared by uniformly and intimately bringing the active compounds into association with a liquid carrier, a finely divided solid carrier, or both, and then, if necessary, shaping the product into desired formulations.

Formulations of the present invention suitable for oral administration may be presented as discrete units such as capsules, cachets, tablets, or lozenges, each containing a predetermined amount of the active ingredient as a powder or in the form of granules; or as a suspension in an aqueous liquor or a non-aqueous liquid, such as a syrup, an elixir, an emulsion, or a draught.

A tablet may be made by compression or molding, optionally with one or more accessory ingredients. Compressed tablets may be prepared by compressing in a suitable machine, with the active compound being in a free-flowing form such as a powder or granules which optionally is mixed with a binder, disintegrant, lubricant, inert diluent, surface active agent, or discharging agent. Molded tablets comprised of a mixture of the powdered active compound with a suitable carrier may be made by molding in a suitable machine.

A syrup may be made by adding the active compound to a concentrated aqueous solution of a sugar, for example sucrose, to which may also be added any accessory ingredient(s). Such accessory ingredient(s) may include flavorings, suitable preservatives, agents to retard crystallization of the sugar, and agents to increase the solubility of any other ingredient, such as a polyhydroxy alcohol, for example glycerol or sorbitol.

Formulations suitable for parenteral administration conveniently comprise a sterile aqueous preparation of the active compound, which preferably is isotonic with the blood of the recipient (e.g., physiological saline solution).

Nasal spray formulations comprise purified aqueous solutions of the active compound with preservative agents and isotonic agents. Such formulations preferably are adjusted to a pH and isotonic state compatible with the nasal mucous membranes.

Formulations for rectal administration may be presented as a suppository with a suitable carrier such as cocoa butter, hydrogenated fats, or hydrogenated fatty carboxylic acids.

Ophthalmic formulations are prepared by a similar method to the nasal spray, except that the pH and isotonic factors are preferably adjusted to match that of the eye.

Topical formulations comprise the active compound dissolved or suspended in one or more media, such as mineral oil, petroleum, polyhydroxy alcohols, or other bases used for topical pharmaceutical formulations.

In addition to the aforementioned ingredients, the formulations of this invention may further include one or more accessory ingredient(s) selected from diluents, buffers, flavoring agents, binders, disintegrants, surface active agents, thickeners, lubricants, preservatives (including antioxidants), and the like.

Cultured cells use the integrin proteins to attach to their growth substrate (in this case plastic flasks). If boric acid is acting at the level of the integrins then cellular attachment should be inhibited in the presence of boric acid. If the binding of boric acid causes the integrins to change their shape, then a larger boric acid derivative (i.e. phenyl boric acid, PBA) should have a more pronounced effect on cell attachment. To measure the effects of boric acid, phenyl boric acid and Manganese on attachment, ZR-55-1 cells were harvested with trypsin and then plated into flasks containing media and these compounds. The table below contains the percentage of cells that had reattached at the times indicated.

TABLE 1
Cell Attachment

| | | | | | |
|---|---|---|---|---|---|
| Exp. | Time (min) | 30 | 60 | 90 | 120 |

ZR-75-1

| | | | | | |
|---|---|---|---|---|---|
| 1 | Control (C) | 23.7 ± 11.9 | 68.5 ± 21.4 | — | 108 ± 28.0 |
| 1 | (C) + 1 mM BA | 24.5 ± 5.2 | 74.1 ± 26.1 | 98.2 ± 34.0 | 104.0 ± 16.1 |
| 1 | (C) + 1 mM PBA | 36.0 ± 11.1 | 38.1 ± 3.8 | 74.7 ± 6.0 | 80.8 ± 16.2 |
| 2 | Control (C) | 27.7 ± 6.8 | 47.9 ± 2.0 | 60.5 ± 5.2 | 69.9 ± 6.9 |
| 2 | C + 1 mM MnCl₂ | 42.6 ± 12.0 | 67.3 ± 12.0 | 70.2 ± 1.4 | 77.8 ± 13.6 |
| 2 | C + 1 mM MnCl₂ + 1 mM PBA | 44.2 ± 4.2 | 49.9 ± 7.1 | 65.8 ± 2.5 | 57.9 ± 11.0 |

MCF-7

| | | | | | |
|---|---|---|---|---|---|
| 3 | Control (C) | 49.7 ± 6.7 | 99.6 ± 7.1 | 108.7 ± 6.8 | 99.3 ± 0.8 |
| 3 | (C) + 1 mM BA | 53.5 ± 3.0 | 92.6 ± 9.1 | 105.7 ± 6.8 | 97.5 ± 4.1 |
| 3 | (C) + 1 mM PBA | 46.3 ± 7.3 | 81.7 ± 2.3 | 99.3 ± 4.3 | 95.0 ± 6.8 |
| 4 | Control (C) | 26.0 ± 0.1 | 52.4 ± 1.9 | 64.5 ± 5.3 | 80.6 ± 4.4 |
| 4 | C + 1 mM MnCl₂ | 53.9 ± 12.3 | 69.4 ± 2.9 | 74.9 ± 3.6 | 81.5 ± 9.9 |
| 4 | C + 1 mM MnCl₂ + 1 mM PBA | 44.7 ± 1.7 | 51.2 ± 3.0 | 68.4 ± 2.8 | 66.1 ± 4.4 |

Experiments 1 & 3 from table 1 indicate that 1 mM PBA inhibits cell attachment but 1 mM BA did not. Experiments 2 & 4 show that 1 mM MnCl₂ stimulates attachment and that 1 mM PBA can inhibit this stimulation.

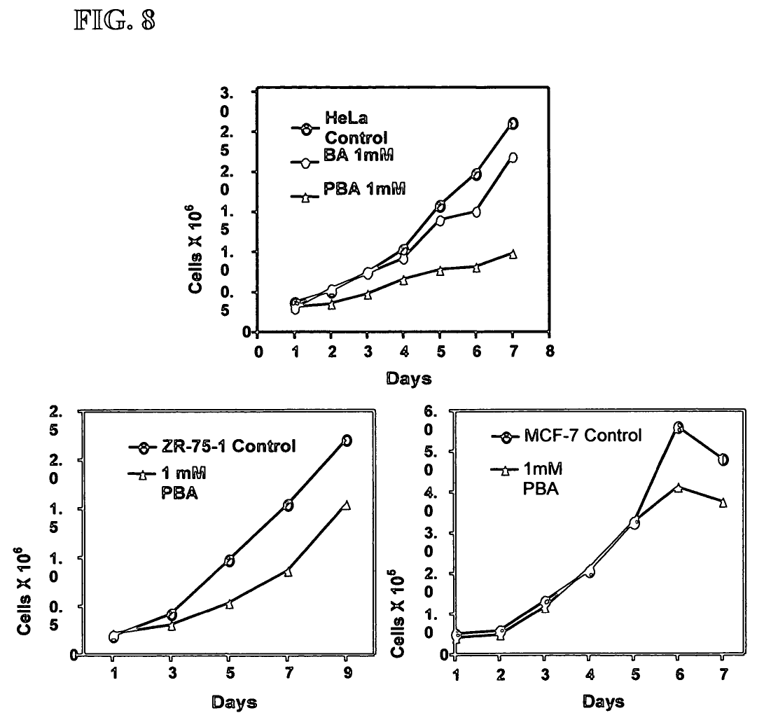

To demonstrate that the effects of boric acid are not due to the presence of the NaBC1 transporter, we cultured HeLa cells (which express the NaBC1 transporter) in the presence of 1 mM boric acid and observed very little inhibition. However, if we grew HeLa cells in the presence of 1 mM phenyl boric acid, a molecule too large to fit through the NaBC1 transporter, significant inhibition was observed (FIG. 8). When ZR-75-1 cells are grown in the presence of 1 mM phenyl boric acid significant growth inhibition is also observed while slight inhibition is seen in the MCF-7 cells (FIG. 8).

We have identified boric acid sensitive and insensitive breast cancer cell lines. Therefore, a fundamental difference in integrin expression (subunit composition, expression levels, surface expression, and integrin activity) should exist between the boric acid responding and non-responding cell lines. In addition, there are two potential boric acid binding locations and both may play a role in the response to boric acid. The first place that boric acid could bind is to the carbohydrate moieties on the glycosylated integrins. The second place that boric acid could bind are the metal ion binding sites on both the α and β chains that make up the integrins. Finally, inhibition of integrin attachment and signaling in the presence of boric acid should lead to the impairment of these cells to migrate.

Since the ZR-75-1 cell line is very sensitive to boric acid inhibition while the MCF-7 cell line is much less sensitive it would stand to reason that there is some difference in expression of a critical target in these cell lines. Since boric acid caused actively growing ZR-75-1 cells to detach and inhibited their reattachment the most likely target is the integrin proteins.

Western blots can be used to determine which set of α and β chains are expressed by both cell lines (ZR-75-1 and MCF-7). From these results we can predict which integrins may be present on the cell surface. For example if MCF-7 cells express α₃ and α₅ as well as β₁ and β₃ chains then the possibility exists that α₃β₂, α₃β₃, α₅β₁, α₅β₃ integrins may be expressed. To confirm which of these integrins are expressed, MAbs to each integrin will be added to intact cells. Most integrins can be detected by a variety of commercially available antibodies. Then a fluorescent secondary antibody to detect the MAbs will be added and the fluorescence detected via flow cytometric methods (57). To confirm and expand these results, microarray analysis will be performed on the cell lines using the CodeLink system.

To confirm that a specific integrin is playing a crucial role in the response to boric acid, a non-responsive cell line MDA-MB-435 will be transfected with the α and β integrin chains from a responsive cell line and growth in the presence of boric acid will be measured.

The most obvious target would be the NaBC1 boron transporter. Microarray analysis should provide several other targets that are differentially expressed between the breast cancer cell lines. The lack of commercially available antibodies would slow down the confirmation of integrin expression. This problem could be solved by obtaining antibodies directly from other researchers or using a company to produce the antibodies needed for the specific integrin detection.

Since integrins play a major role in the migration of cells it is reasonable to assume that borates would inhibit migration if the major target of borate binding are the integrins. Borates could also inhibit migration by altering the rate of integrin recycling so that rate will also be measured.

The last specific aim of investigation would use a standard migration assay. Cells would be grown in the presence of BA or PBA and then harvested for the migration assay. Approximately 250,000 cells would be loaded into the upper migration chamber of a Corning transwell permeable support (24-well transwell, 8 μm polycarbonate membrane) in 0.1 ml of serum-free RPMI-1640 media. The lower chamber would be contain 0.6 ml of RPMI supplemented with 10% FBS to serve as a chemo-attractant. Plates would be covered and incubated for 24 hrs at 37° C., 5% CO₂. Following incubation, cell remaining of the upper filter would be removed with a cotton swab, while the migrated population on the filter side will be washed with PBS, fixed in methanol, stained and counted.

To evaluate the role that borates may play in inhibiting integrin recycling pulse-chase experiments in the presence and absence of borates would be conducted. Cells would be incubated with anti-integrin Fab fragments (0.5 g/ml) for 30 min at 37° C. to allow for optimal internalization of the integrin-Fab complex. To avoid internalization induced by cross-linking caused with bivalent antibodies, Fab fragments are routinely used to follow receptor internalization and trafficking. The cells would then be cooled to room temperature to stop further endocytic trafficking and would be incubated with unlabeled anti-mouse IgG (1:100; Jackson ImmunoResearch Laboratories) for 30 min at room temperature to block the Fab remaining at the cell surface. After gentle washing, the cells would be reincubated at 37° C. for 20 or 90 min in media to allow for trafficking of endocytosed integrin-Fab complexes. The cells would then be fixed with 2% PFA with 30% sucrose for 20 min at room temperature, and incubated with an anti-actin antibody to reveal whether the cells had been permeabilized during fixation. Cells treated with 0.1% Triton X-100 (22686; USBiological) would be used as permeabilization controls. Cells fixed but not permeabilized before the reincubation period would be used as 0-min time points. The recycled integrin on the cell surface would be detected by visualizing the anti-integrin Fab that had not been blocked with the unconjugated secondary. The unpermeabilized cells would be incubated with Alexa-conjugated secondary antibodies (1:500; Molecular Probes, Inc.) for 1 hr at room temperature.

The technology of the present description may be practices as a method for the treatment or prevention of breast cancer cell lines. The boron compound, and especially the boric acid solution or boric acid salt may be provided in liquid or solid form. For example, this can be done as providing boric acid or boric acid salts to the patient in a manner that delivers boric acid or boric acid salt to breast cancer cells and the breast region of a patient. The method may be for the treatment of breast cancer cell lines selected from identified groups previously identified as being growth reduction affected by boric and its salts and the method comprises, as by identifying the presence in a patient of at least one of breast cancer cell lines of the identified groups in a patient; and providing a solution of boric acid or boric acid salts to the patient in a manner that delivers boric acid or boric acid salt to breast cancer cells of the identified groups in the patient. The method may perform the process for the prevention of breast cancer cell lines where the boron compounds, such as boric acid salts, are provided in solid, capsule, caplet, pill, or other orally ingestible form and orally ingested by the patient.

Other variations in concentrations, delivery systems, adjuvants and the like may be used in the practice of the present technology without deviating from the generic nature of the teachings herein.

APPENDIX I
LIST OF REFERENCE SOURCES FOR POTENTIAL BREAST CANCER CELL
LINES WHERE TREATMENT ACORDING TO THE DISCLOSED TECHNOLOGY
MAY ALSO BE EFFECTIVE:

| Gene | Map | Methylated | Notes | Accession # |
|---|---|---|---|---|
| 14-3-3 Sigma | 1p | Breast and gastric cancers | | AF029081 |
| ABL1 (P1) | 9q34.1 | 50-100% CML, Some ALL | Only methylated when part of | M14752 |
| | | | the bcr-abl translocation. | |
| ABO | 9q34 | cell lines | — | NM_020469 |
| APC | 5q21 | Colon, gastric and | One promoter only. Correlation | NM_000038 |
| | | esophageal cancer | with expression not established. | |
| | | | Type A | |
| AR (Androgen | Xq11-12 | Prostate Cancer Cell Lines, Colon ACFs | | NM_000044 |
| Receptor) | | | | |
| BLT1 | | Various cell lines | — | D89079 |
| (Leukotriene B4 | | | | |
| Receptor) | | | | |
| BRCA1 | 17q21 | 10-20% Breast cancer, | Cause of transcriptional | AF274503 |
| | | some ovarian | silencing in these cells | |
| CALCA | 11p15 | 25-75% Colon, lung, | One of the first promoter-CpG | M64486 |
| (Calcitonin) | | hematopoic neoplasms. | islands demonstarted to be | |
| | | | hypermethylated in cancer. | |
| CASP8 (CASPASE | 2q33-34 | Neuroblastoma | Corralates with MycN | NM_001228 |
| 8) | | | amplification | |
| Caveolin 1 | 7q31.1 | Breast cancer cell lines | | NM_001753 |
| CD44 | 11pter-p13 | Prostate cancer | | AJ251595 |
| CDH1 | 16q22.1 | Herediatory gastric cancer | methylation as a second hit | L34936 |
| CFTR | 7q31.2 | Cell Lines | No primary tumors reported | NM_000492 |
| GNAL | | | | NM_002071 |
| COX2 | 1q25.2-q25.3 | Colon, Breast and prostate | Correlates with expression when | 10701070[EST] |
| | | cell lines. 15% of primary | completely methylated. | |
| | | colon cancers | | |
| CSPG2 (Versican) | 5q12-14 | Aging Colon. 70% colon | Secreted proteoglycan, | NM_004385 |
| | | | regulated by Rb. | |
| CX26 (Connexin | 13q11-q12 | Breast cancer cell lines | No correlation with expression | NM_004004 |
| 26) | | | | |
| Cyclin A1 | 13q12.3-q13 | Various cell lines | | NM_003914 |
| DAPK1 | | | | XM_005442 |
| DBCCR1 | 9q32-33 | 50% Bladder cancer | Slight methylation in normal | NM_014618 |
| | | | bladder aging-related | |
| DCIS-1 | | Ductal Carcinoma | | L27636 |
| ECAD (E- | 16q22.1 | 20-70% Breast, Gastric, | Methylation is often | D49685 |
| cadherin) | | Thyroid, SCC, Leukemias | heterogeneous and not always | |
| | | and Liver ca. | correlated with silencing. Also | |
| | | | present in some normal stomach | |
| | | | and liver samples (Aging). | |
| Endothelin | 13q22 | 60-70% prostate cancer | — | NM_005302 |
| Receptor B | | | | |
| EPHA3 | 3p11.2 | Leukemias | | NM_005233 |
| EPO | 7q21 | HeLa Cells | Normal and primary tumors | AF202312 |
| Erythropoietin) | | | | |
| ER (Estrogen | 6q25.1 | Aging colon, liver, heart | Upstream promoter not in CpG | X62462 |
| Receptor) | | muscle, AoSMC (cultured), | island. Nevertheless, there is a | |
| | | ?brain, Not breast/lung | good correlation with loss of | |
| | | epithelium, AoEC. 100% | expression. | |
| | | Colon cancer 20-30% ER- | | |
| | | breast cancer 60-70% | | |
| | | AML/ALL 20-50% CML-BC | | |
| | | 20% Lung (NSCLC) 60% GBM | | |
| FHIT | 3p14.2 | 10-20% Esophageal SCC | — | NM_002012 |
| GALNR2 | 17q25.3 | | | AF058762 |
| GATA-3 | | | | X55122 |
| COL9A1 | 6q12-q14 | | | M32133 |
| GPC3 (Glypican 3) | Xq26 | Mesothelioma and Ovarian cancer cell lines | | NM_016697 |
| GST-pi | 11q13 | 80-100% Prostate, Liver. | DNA repair/detoxification | M37065 |
| | | 30-60% Colon, Breast, | enzyme. | |
| | | Kidney. | | |
| GTP-binding | | | | L10665 |
| protein (olfactory | | | | |
| subunit) | | | | |
| H19 | 11p15.5 | 20-50% Wilm's tumors | Imprinted gene. | AF087017 |
| | | | Hypermethylation is associated | |
| | | | with apparent loss of imprinting | |
| | | | of the IGF2 gene in Wilm's | |
| | | | tumor, but not others. | |
| H-Cadherin | 16q24.1-24.2 | 45% Lung Cancer, some | — | AB001090 |
| (CDH13) | 24.2 | ovarian cancer | | |
| HIC1 | 17p13.3 | Aging Prostate, ?Breast and | Candidate tumor-suppressor | NM_006497 |
| | | Brain, NOT Colon. 80-100% | gene. First gene cloned based | |
| | | Colon cancer, Prostate, | on finding a CpG island | |
| | | Breast, GBM. 20-50% Lung, | hypermethylated in cancer. | |
| | | Kidney, Liquid tumors. | | |
| hMLH1 | 2p22 | 10-20% colon, endometrial | Almost always associated with | AB017806 |
| | | and gastric cancers. 0% | microsatellite instability and, in | |
| | | lung, breast, GBM, liquid | celllines, mismatch repair | |
| | | tumors etc. | deficiency. | |
| HOXA5 | 7p15-p14.2 | Breast cancer | | NM_019102 |
| IGF2 (Insulin-Like | 11p15.5 | AGING colon 100% Colon | IGF2 has a large CpG island that | NM_000612 |
| Growth Factor II) | | cancer 50% AML | contains the imprinted P2-4 | |
| | | | promoters but NOT the non- | |
| | | | imprinted P1, which is | |
| | | | unaffected by this | |
| | | | hypermethylation. | |
| IGFBP7 | 4q12 | Murine SV40 T/t antigen- | ? Normal and primary tumors | NM_001553 |
| | | induced | | |
| | | hepatocarcinogenesis | | |
| IRF7 | 11 | Various cell lines | | NM_001572 |
| KAI1 | | | | AF081565 |
| LKB1 | 19p13.3 | A few colon, testicular and breast (medullary) primary | | AF035625 |
| | | tumors | | |
| LRP-2 (Megalin) | 2q24-31 | Various cell lines | | AF065440 |
| MDGI (Mammary- | 1p35-33 | 50-70% Breast cancers | — | Y10255 |
| derived growth | | | | |
| inhibitor) | | | | |
| MDR1 | 7q21.1 | Drug sensitive leukemia | ?Primary tumors | NM_000927 |
| | | cell lines. | | |
| MDR3 (PGY3) | 7q21.1 | Various cell lines | | Z35286 |
| MGMT (O6 methyl | 10q26 | 25-50% Brain, colon, lung, | Associated with the MER- | M29971 |
| guanine methyl | | breast, NHL etc. | phenotype | |
| transferase) | | | | |
| MINT | | | | AF135501 |
| MT1a | 16q13 | Rat hepatoma | ? Normal and primary tumors | K01383 |
| (metallothionein | | | | |
| 1) | | | | |
| MUC2 | 11p15.5 | Colon cancer cell line | ?Primary tumors | NM_002457 |
| MYOD1 | 11p15.4 | AGING Colon. 100% colon, | One of the first promoter-CpG | NM_002479 |
| | | 30% breast, Also bladder, | islands demonstarted to be | |
| | | lung, liquid tumors. | hypermethylated in cancer. | |
| N33 | 8p22 | Aging Colon. 60-80% colon, | Oligo-saccharyl-transferase | NM_006765 |
| | | prostate, brain. | | |
| NEP (Neutral | 3q21-27 | Prostate cancer (˜10%) | | NM_000902 |
| Endopeptidase | | | | |
| 24.1)/CALLA | | | | |
| NF-L (light- | 8p21 | Rat Glioma cell line | No primary tumors reported | S70309 |
| neurofilament- | | | | |
| encoding gene) | | | | |
| NIS (sodium- | 19p13.2-p12 | Thyroid cancer cell lines | Heterogeneous methylation in | AF260700 |
| iodide symporter | | | primary tumors | |
| gene) | | | | |
| “OCT-6” | | | | L26494 |
| P14/ARF | 9p21 | Colon cancer cell lines | Less frequent than P16 | AK024826 |
| | | (infrequent) | methylation, but usually | |
| | | | associated with P16 | |
| | | | methylation. | |
| P15 (CDKN2B) | 9p21 | 80% AML/ALL 2-20% GBM | P15 is physically close to P16, | AF058758 |
| | | 0% Colon/Lung/Breast | but simultaneous methylation of | |
| | | | both genes is rare. | |
| P16 (CDKN2A) | 9p21 | 20-30% Lung (NSCLC) 25-35% | Methylation is as frequent as | NM_000077 |
| | | Colon 5-25% | deletions, and more frequent | |
| | | Lymphomas (depending on | than mutations. P19 (alternate | |
| | | stage) 0-5% Bladder. Many | first exon and reading frame) is | |
| | | others (esophagus, | not methylated in cancer. | |
| | | stomach etc.) | | |
| P27KIP1 | 12p13 | Rodent pituitary cancer | No primary tumors reported | AY004255 |
| | | cell lines | | |
| p57 KIP2 | 11p15.5 | Gastric cancer cell lines | | AC005950 |
| p73 | | | | AF276941 |
| PAX6 | 11p13 | Colon cancer cell lines and 70% of primary tumors | | NM_000280 |
| PgR | 11q22 | 10-20% Breast cancer | Effect on transcription | NM_000926 |
| (Progesterone | | | | |
| Receptor) | | | | |
| POU3F1 | | | | NM_002699 |
| RAR-Beta2 | 3p24 | Colon, Breast, Lung Cancer | | X56849 |
| RASSF1 | 3p21.3 | Lung cancer | 1 promoter only | AF291719 |
| RB1 | 13q14 | 10-20% Retinoblastomas 0% | Does anyone know whether a | NM_000321 |
| (Retinoblastoma) | | Lung/Leukemia/Colon | correlation with expression has | |
| | | Some pituitary adenomas | been established? | |
| RPA2 (replication | | | | NM_002946 |
| protein A2) | | | | |
| SIM2 | | | | D85922 |
| TERT | 5p15.33 | Heterogeneous methylation in many cell lines | | AB018788 |
| TESTIN | 7q31.2 | Hematopoietic | One promoter only | NM_015641 |
| | | malignancies | | |
| TGFBR1 | 9q33-q34 | Gastric cancer cell lines and primary tumors (10%) | | AF054590 |
| THBS1 | 15q15 | 5-10% Colon Cancer 30-40% | Angiogenesis inhibitor, | NM_003246 |
| (Thrombospondin- | | GBM 20-30% AML 0% | regulated by P53 and Rb in some | |
| 1) | | Endometrial/Breast | systems. | |
| TIMP3 | 22q12.1-13.2 | Human brain (10-50%) and kidney (20%) cancers, Mouse | | NM_000362 |
| | | model | | |
| TLS3 (T-Plastin) | X | Leukemia cell lines | | NM_005032 |
| TMEFF2 | | | | NM_016192 |
| Urokinase (uPA) | 10q24 | Breast cancer cell lines | | AF107292 |
| VHL (Von-Hippell | 3p25-25 | 10-20% Renal Cell cancers | Same tumor selectivity as | NM_000551 |
| Lindau) | | 0% Common solid and | mutations | |
| | | liquid tumors | | |
| WT1 | 11p13 | 90% Breast cancers, 20-50% | Correlation with expression | X77549 |
| | | colon, 5-10% Wilm's | | |
| ZO2 (Zona | | Pancreatic cancer cell | | 777454 [EST] |
| Occludens 2) | | lines | | |

Specifically Listed Genes: ZNF217; BRCA-1

Recently, scientists have begun to isolate genes responsible for hereditary breast cancer. In 1994 the gene, named Breast Cancer 1 (BRCA-1), was finally isolated in Chromosome #17, one of the 23 pairs of chromosomes found in most human cells. An altered BRCA-1 has been linked to the development of breast and ovarian cancer. In 1995, scientists developed experimental tests for detecting several recently discovered cancer genes, including BRCA-1. However preliminary studies have shown that testing positive for an altered BRCA-1 gene does not necessarily mean a woman will develop breast cancer. At least 15% of the women who carry the altered gene will never develop the disease. Scientists have no way of knowing yet which women fall into that category. In addition, because BRCA-1 alterations occur in many different places scattered throughout the gene, developing an accurate test will be very difficult to do. The altered BRCA-1 gene appears in only 5% of the 182,000 breast cancer cases that develop. If a woman tests negative (that is, she does not have the altered gene), this does not necessarily mean she will be free of breast cancer during her lifetime.

BRCA-2—The gene BRCA-2 on Chromosome #13. Like BRCA-1, BRCA-2 appears to be a cancer-causing gene when altered. BRCA-2 appears to account for as many cases of breast cancer as does BRCA- 1. BRCA-2 apparently triggers breast cancer in males as well as in females.

P53—There are specific genes in the cells of human bodies that normally help to prevent tumors from forming. One of these tumor-suppressor genes, called P53 (“p” for protein and “53” for its weight) was recently named “Molecule of the Year” by the editors of the journal Science. This protein plays a major role in cell growth. The job of P53 is to prevent (suppress) cells from growing. When it has been damaged or altered, P53 loses its ability to block cell growth. Changes to the gene result in an increased risk of cancer. Almost 50% of all human cancer cells contain a P53 mutation. These cancers are more aggressive and more often fatal. Since P53 is so important for normal cell growth in humans, researchers are continuing to look for ways to diagnose, prevent, and treat cancer associated with P53.

ATM—After more than a decade of intensive searching, researchers have isolated a recessive gene that increases the risk for people to develop some kinds of cancer (as well as a rare genetic disease). The gene, ataxia telangiectasia mutated (ATM) may be involved in many cancers, including breast cancer. The normal role of the ATM gene is to control cell division. Although researchers do not know why an altered ATM causes cancer, 1% of Americans (more than 2 million people) carry at least one copy of the defective form of the gene. By examining the role of altered ATM genes, scientists are hoping to shed some light on what makes cells live, grow, and die. Besides being associated with cancers, the ATM gene may also identify those individuals who are sensitive to radiation. The altered form of the ATM gene is closely linked to a childhood disorder of the nervous system called Ataxia Telangiectasia, or AT. AT afflicts 1 in 40,000 children in the U.S. and 1 in 200,000 worldwide each year.

P65—With the recent discovery of the gene called P65, scientists are hoping to develop a blood test to detect cancers of the breast and prostate at a much earlier stage than is now possible. The altered form of P65 is linked to the overproduction of certain hormones that may help to cause both breast and prostate cancers. The new blood test, called the tumor blood marker, hopefully will allow doctors to monitor a patient's response to cancer treatment. The level of the P65 protein marker in the blood decreases as tumors are destroyed during therapy. A study is being performed to determine if the tumor marker blood test is suitable for widespread use. Human breast tumors are diverse in their natural history and in their responsiveness to treatments. Variation in transcriptional programs accounts for much of the biological diversity of human cells and tumors. In each cell, signal transduction and regulatory systems transduce information from the cell's identity to its environmental status, thereby controlling the level of expression of every gene in the genome.

What is claimed:

1. A method for the treatment of breast cancer cell lines selected from the group consisting of ZR-75-1 and SK-BR-3, the method comprising:
identifying the presence of at least one of breast cancer cell lines ZR-75-1 and SK-BR-3 in a patient; and
providing a solution of boric acid or boric acid salts to the patient in a manner that delivers boric acid or boric acid salt to breast cancer cells in the patient.

2. The method of claim 1 wherein the solution comprises at least 0.05 mM of boric acid or boric acid salt.

3. The method of claim 1 wherein the solution comprises at least 0.10 mM of boric acid or boric acid salt.

4. The method of claim 2 wherein the solution is delivered intravenously.

5. The method of claim 3 wherein the solution is delivered intravenously.

6. The method of claim 2 wherein the boric acid is injected to a site where breast cancer cells have been located within the patient.

7. The method of claim 3 wherein the boric acid is injected to a site where breast cancer cells have been located within the patient.

8. A method for the treatment or prevention of breast cancer cell lines comprising
providing boric acid or boric acid salts to the patient in a manner that delivers boric acid or boric acid salt to breast cancer cells and the breast region of a patient.

9. The method of claim 8 wherein the method is for the treatment of breast cancer cell lines selected from identified groups previously identified as being growth reduction affected by boric and its salts and the method comprises:
identifying the presence in a patient of at least one of breast cancer cell lines of the identified groups in a patient; and
providing a solution of boric acid or boric acid salts to the patient in a manner that delivers boric acid or boric acid salt to breast cancer cells of the identified groups in the patient.

10. The method of claim 8 wherein the process is for the prevention of breast cancer cell lines and the boric acid salts are provided in solid form and orally ingested by the patient.

11. The method of claim 1 wherein the boric acid or boric acid salt is assisted in cell delivery by the addition to a patient of an effective amount of at least one ion transporting protein.

12. A method for the treatment of breast cancer cell lines selected from the group consisting of ZR-75-1 and SK-BR-3, the method comprising:
identifying the presence of at least one of breast cancer cell lines ZR-75-1 and SK-BR-3 in a patient; and
providing a solution of organic boron compound to the patient in a manner that delivers the organic boron compound to breast cancer cells in the patient.

13. The method of claim 10 wherein the organic boron compound is assisted in cell delivery by the addition to a patient of an effective amount of at least one ion transporting protein.

14. The method of claim 11 wherein the organic boron compound comprises a borane or borane salt.
